(19) United States
(12) Patent Application Publication
(10) Pub. No.: US 2004/0234618 A1
(43) Pub. Date: Nov. 25, 2004

(54) Method and an apparatus for energizing fluids

(76) Inventor: Thomas Saito, Newmarket (CA)

Correspondence Address: Schwegman Lundberg Woessner & Kluth, P O Box 2938, Minneapolis, MN, 55402, US

(21) Appl. No.: 10/469,577

(22) PCT Filed: Feb. 1, 2001
(86) PCT No.: PCT/SE01/00198

(51) Int. Cl. A61K 33/00; G01N 27/26

(52) U.S. Cl. 424/600; 204/400

(57) ABSTRACT

The invention relates to a method and an apparatus for energizing fluids, comprising the step of subjecting a starting liquid to a process in order to produce an energized liquid the molecules of which are clustered and have a specific wavelength. In accordance with the invention the method also includes the steps of mixing the energized liquid with grains of a memorizing mineral so as to not only add to the liquid trace amounts of the mineral of said grains, but also subjecting the grains themselves to an energizing action by the clustered and structured liquid, thereby memorizing the structure and/or energy level of said liquid in said grains. Thereafter the memory carrying grains are separated from said liquid and preferably dried in order to make the grains fit for energizing other fluids.

### TECHNICAL FIELD OF THE INVENTION

In a first aspect this invention relates to a method for energizing fluids, comprising the step of subjecting a starting liquid to the action of an electrical current, and/or a magnetic field and/or a pulsed colour light, and/or a pulsed red laser light so as to produce an energized liquid, the molecules of which are clustered and have a specific wave length.

### PRIOR ART

In almost every system on earth consisting of, or incorporating, biological substances, water is the base or carrier of different functions and processes. Natural liquid water has an unpredictable structure consisting of both unbonded and disordered water molecules connected to others related as bonded internal and surface clusters, all connected to each other by hydrogen bonding. Water is a non-reproducible resource and due to mankind's more and more extensive use of the nature the pressure to the environment stresses the water structures even more. Water clusters are a natural phenomenon itself, but the size and structure of the natural clusters can not be predicted. The possibility of artificially energizing the water by forming a specified high-quality and replicable water cluster structure is therefore attractive for a variety of practical applications.

In the past a number of attempts have been made to produce clustered water artificially. Thus U.S. Pat. No. 5,711,950 discloses a process for preparing clustered water inter alia comprising the steps of boiling water to produce steam and passing the steam across a magnetic field as well as exposing the steam to light having a wave length of between 610 nm and 1 mm. This process is however rather complicated and expensive, and not suitable for producing clustered water industrially on a large scale.

### OBJECTS AND FEATURES OF THE PRESENT INVENTION

The present invention aims at overcoming the shortages of the prior technique and providing an improved method for producing clustered energized fluids in general. Therefore, a primary object of the invention is to provide a method by which large quantities of energized fluids having specified energy levels can be produced artificially for a variety of practical applications. A further object of the invention is to provide a method which makes the practical energization of different fluids easy to realize.

In accordance with the invention the above-mentioned objects are attained by the steps of mixing a specifically energized and clustered liquid with grains of a memorizing mineral so as to not only add to the liquid trace amounts of the mineral of said grains thereby structuring molecular chains of the the clustered liquid, but also subjecting the grains themselves to an energizing action by the clustered and structured liquid, thereby memorizing the structure and/or energy level of said liquid in said grains, and separating said memory-carrying grains from said liquid in order to make said grains fit for energizing other fluids.

In a second aspect the present invention also relates to apparatus for energizing fluids, i.e. for realizing the method. The features of the apparatus are evident from claims 5 to 7.

### BRIEF DESCRIPTION OF THE ATTACHED DRAWINGS

In the drawings:

FIG. 1 is a schematic illustration of a basic apparatus for producing energized fluids and memory-carrying grains in accordance with the invention,

FIG. 2 is an axial section through an apparatus for the final treatment of a fluid, using energized grains of the type produced in the apparatus of FIG. 1,

FIG. 3 is a section of a cassette-like tube included in the apparatus of FIG. 2,

FIGS. 4-7 photographic illustrations of different types of water; FIGS. 6 and 7 illustrating examples of the structure of a water treated in accordance with the invention, and

FIGS. 8-11 diagrams showing frequencies of the different types of water illustrated in the photos of FIGS. 4-7.

### DETAILED DESCRIPTION OF THE INVENTION

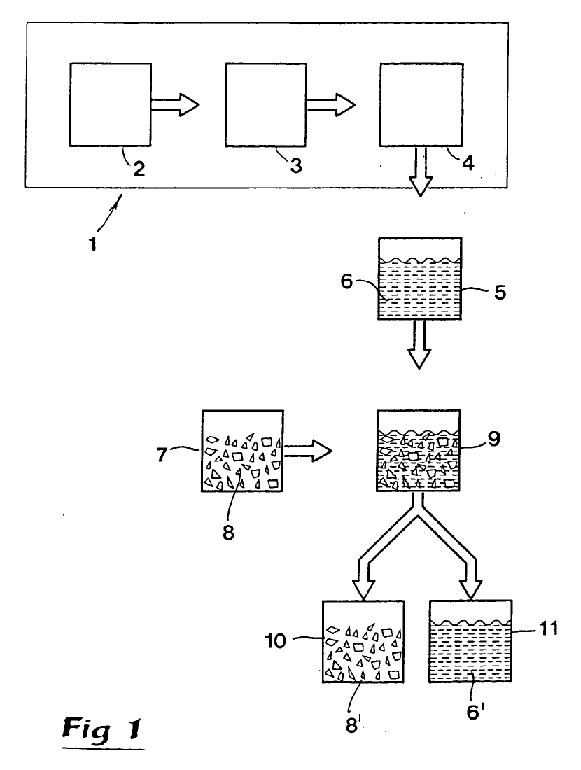

In FIG. 1 reference numeral 1 generally designates an apparatus or assembly including different means for treating a starting liquid in order to cluster the molecules thereof. As a starting material water, e.g. distilled water, may be used. Therefore, the invention will be described hereinafter with reference to water. It is, however, to be understood that the invention may be applied to other arbitrary liquids as well.

In apparatus 1 there is at least a first energy source 2 for clustering the starting water. This source may be a source of electric energy for subjecting the water to the action of an electrical current. It may also be a magnetizer for subjecting the water to a magnetic field. Another alternative is a device for emitting to said water a pulsed colour light. Reference numeral 2 may also indicate a laser for subjecting the water to a pulsed laser light. In this connection it should be emphasized that a combination of two or more of these treatment steps may be used.

Reference numeral 3 designates a transmitter consisting for instance of a rose quartz crystal (e.g. of pyramidic structure) or similar mineral, which is charged with energy from the source 2. In other words electric, magnetic and/or optical energy (one or more in combination) is charged from transmitter 3. The achieved electric, magnetic and/or optical energy is transmitted via a system 4 of certain containers used separately or in combination with other containers and containing either one of the following material: Bach flower remedies, rescue remedies, antioxidants, a combination of vitamins, enzymes, vegetable oils, a combination of minerals, silver, gold, platinum, germanium and/or clustered water. To the container system 4 is connected an output system (not shown) including an electric wire outlet by which the achieved energy levels/rates may be transferred when the outlet is connected to a container for an untreated starting liquid. This container is shown at reference numeral 5, the starting liquid being designated 6. This liquid may be a water, such as distilled water, though it may also be another liquid. The container 5 serves as a store of the liquid to be treated and may be in the shape of a tank.

In another tank or container 7 mineral grains 8 are stored. The mineral in these grains 8 is of a type that can memorize wave lengths. In practice piezoelectric quartz is advantageously used as a mineral.

9 designates another container or vessel in which liquid 6 from tank 5 may be mixed with grains 8 from container 7 and stored together. By apparatus 1 the liquid 6 has been clustered, i.e. energized to a certain energy level. In other words the molecules of the fluid 6 has a well defined frequency being unique for the treatment performed in apparatus 1. The bondage of the molecules in liquid 6 is strong, thus enhancing the quality of the liquid.

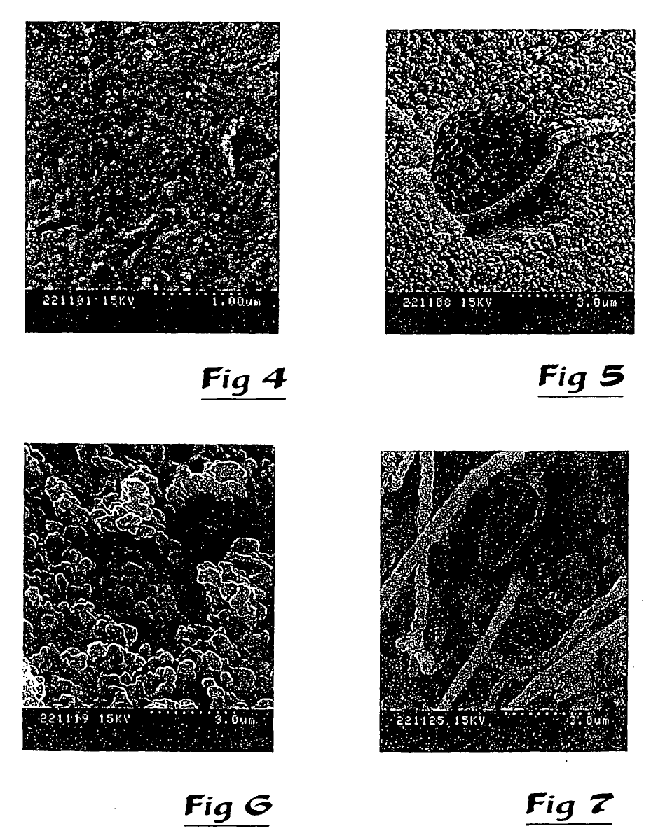

In FIG. 4 the molecule structure of ordinary tap water is photographically shown. FIG. 5 shows the molecule structure of distilled water. In both cases the structure is rather disordered.

FIG. 6 illustrates the molecule structure of clustered water, more specifically water being present as the starting liquid 6 in container 5. Mixing and storing such water 6 and the grains 8 of piezoelectric quartz together in container 9 results in the molecule structure shown in FIG. 7. When grains 8 are mixed with water 6 two phenomena occur. First of all trace amounts of the mineral, i.e. the piezoelectric quartz, are added to the water. Secondly the quartz grains, which have a memorizing capability, will be “programmed” with the energy level and/or specific frequency of the water. The mechanism of on one hand the action of the mineral on the water, and on the other hand the programming action of the water on the mineral grains, is not known in detail, but tests performed by the inventor have shown that the well-defined molecule structure according to FIG. 7, which is achieved in container 9, is transferred to the memory of the mineral grains.

In order to attain the desired effect of the measure of mixing the grains and the water, these components should be stored together for a certain time. More precisely said components should be kept together in container 9 for at least 24 hours, preferably at least one week or more.

After a sufficient storing time the grains and the water are separated from each other as indicated by the containers 10 and 11 in FIG. 1. This separation may be carried out by means of a strainer (not shown). After the separation the energized water 6′ in container 11 may be used itself for commercial purposes. The grains 8′ in container 10 may in turn be dried in any suitable manner before being used commercially.

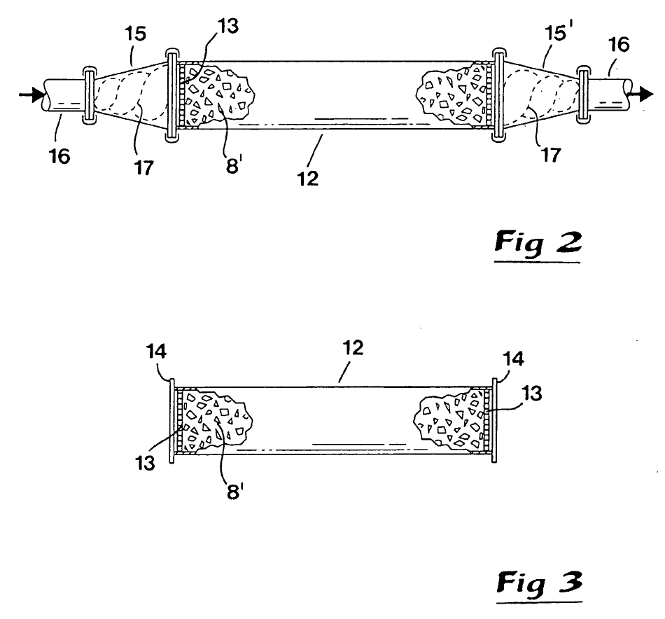

According to the invention the structured cluster of the water 6′ (or liquid) obtained from the treatment not only by apparatus 1, but also by the grains 8, and the unique spectrum of this water cluster structure, can be copied by the mineral grains 8′ so as to transfer this energy to other arbitrary liquids or fluids. Such a transfer can be carried out in accordance with the embodiment shown in FIGS. 2 and 3. These figures illustrate an apparatus including a cassette-like tube or container 12 the interior of which contains a batch of mineral energized grains 8′. The opposite ends of the tube are open in order to allow passages of a fluid through the tube. In order to confine the grains 8′ in the tube there are, however, two strainer walls 13, the holes of which are too small to allow passages of the grains, but allow passage of a fluid. Furthermore tube 12 includes end flanges 14 by means of which the tube can be mounted as shown in FIG. 2. More specifically the tube is mounted as a cassette between a first cone shaped portion 15 of a fluid inlet conduit 16, and a second cone shaped portion 15′ of a fluid outlet conduit 16′. The tube, the cone shaped portions and the conduits my be interconnected by flange connections as indicated in the drawing. In the interior of the cone portions 15, 15′ there are flow converting devices 17, 17′. One of these devices, viz. device 17, on the upstream side functions as a rotation generator, which has the purpose of transforming an axially arriving water flow into a rotating flow or vortex flow in tube 12. Device 17 may consist of a set of screw-shaped blades. Converting device 17′ on the downstream side has the purpose of gradually transposing the rotating flow of the liquid to an axial flow.

By passing a fluid (e.g. a liquid or a gas) through tube 12 the cluster or molecule structure memorized in grains 8′ is transmitted to the fluid.

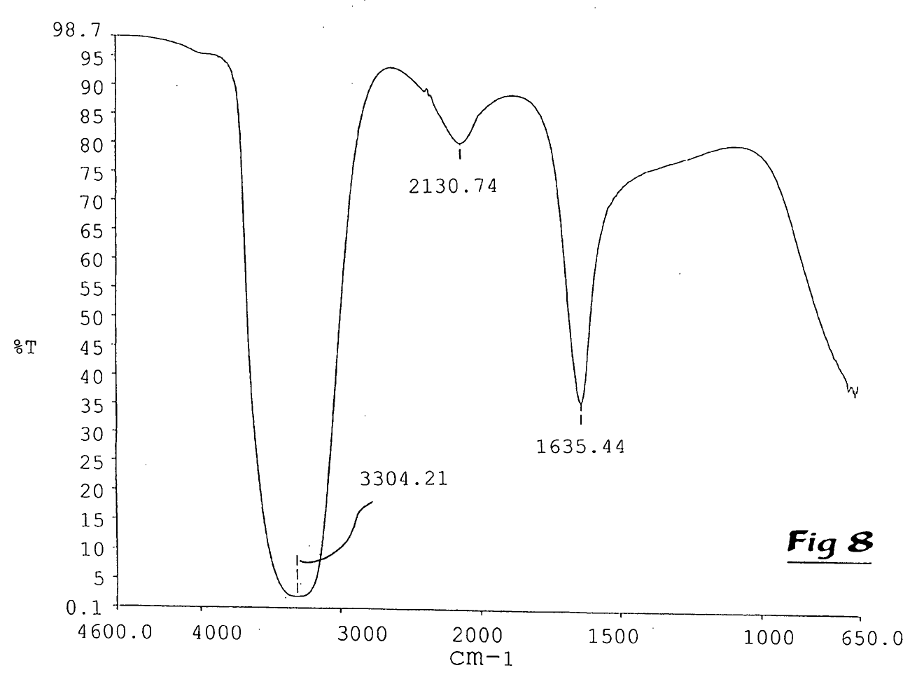

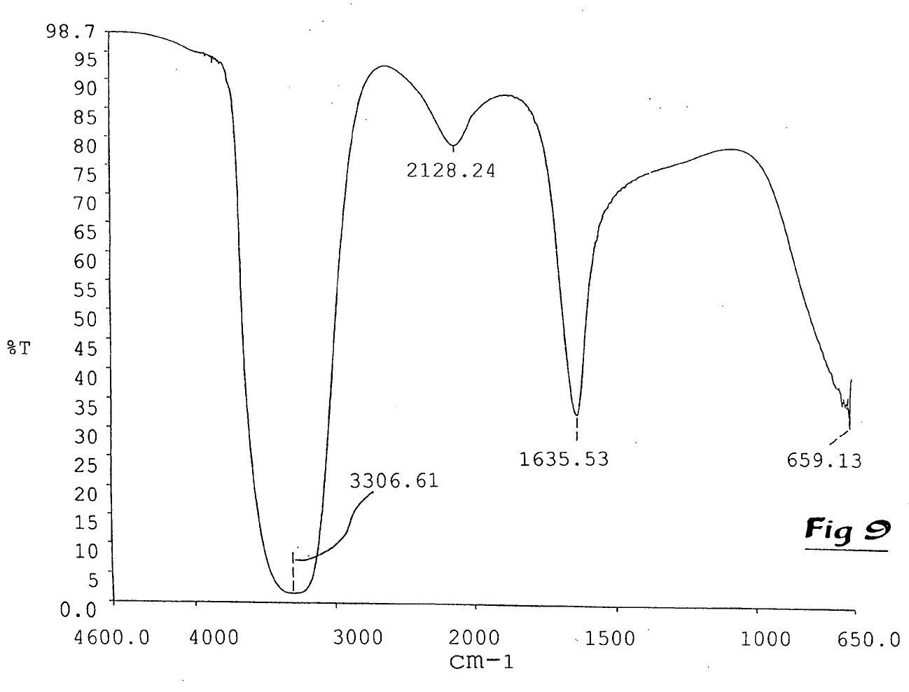

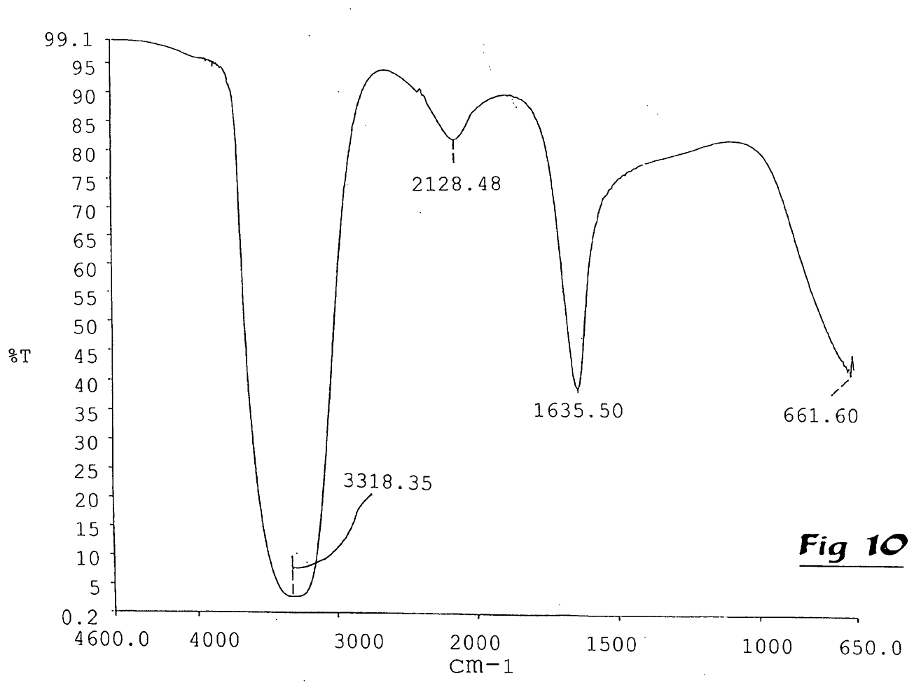

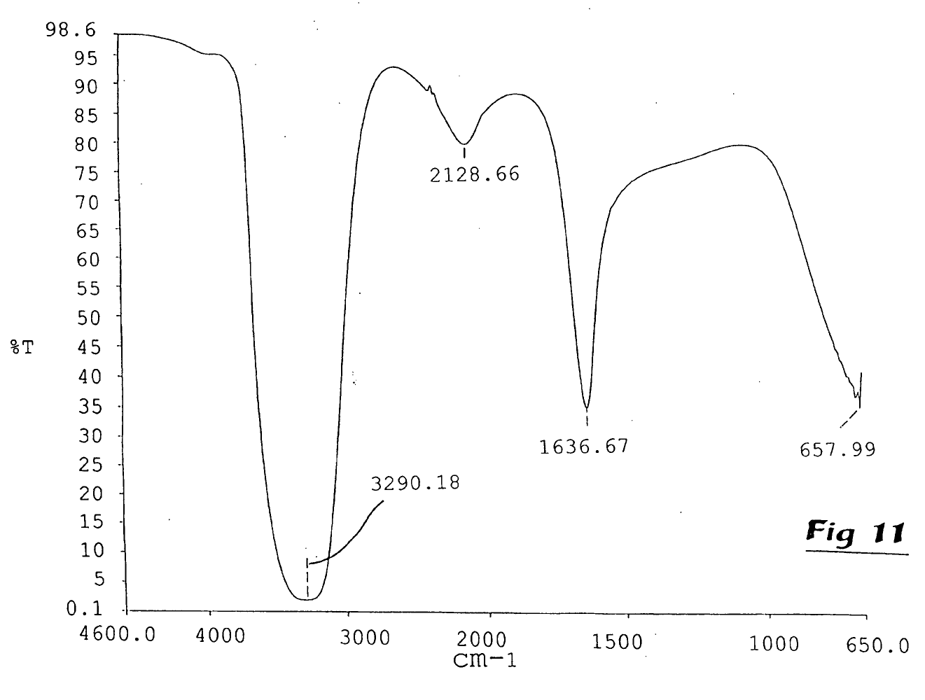

Reference is now made to FIGS. 8-11 which illustrate different frequency characteristics of the different types of water shown in the photos of FIGS. 4-7. The diagrams are of the FTIR-type (fourier transform infrared spectroscopy), the wave number being shown in cm 1 along the X-axis, while the transmittance percentage % T is shown along the Y-axis. The diagram of FIG. 8 shows the frequency characteristics of ordinary tap water of the type shown in the photo of FIG. 4. In a similar manner, the diagram of FIG. 9 shows the frequency characteristics of distilled water of the type shown in FIG. 5. FIGS. 10 and 11 show corresponding diagrams of the frequency characteristics of the clustered water treated in accordance with the invention and shown in the photos of FIGS. 6 and 7 respectively.

By treating fluids in accordance with the present invention, the molecular structure can be clustered at a preset energy level. Frequencies between 1566 and 3 207 168 kHz are obtained and in this range, a bigger number of rates are possible to use in order to get an appropriate energy cluster.

Industrially the apparatus of FIGS. 2 and 3 may advantageously be used for treating liquids, such as water for the production of beverages in a closed system. The invention may, however, be used in many other ways as exemplified below.

A clustered and structured water may be used to enhance the quality and shelf life of many products used for medical care and food production. The programmed energy of the treated water may be added to protein products for human and animal consumption. It may also be used in concentrated health drops, for the dilution of drinking water/beverages, with or without other additives like different herbs, antioxidants, etc. Furthermore it may be used in eye drops as well as in other medical products in order to improve the potency and specificity of the medication. In doing so the specific energy profile of the treated water or protein will activate the wave-length characteristics of the treatment process in the specific region of the human or animal targeted. The high potency of the products is due to the use of the highly specified energy profile, which will control that only the desired medical process is activated. In the medical field the invention may also be utilized for releasing specific neuro transmitters in the brain, for releasing neuro muscular chemicals in muscles, for controlling pain and for releasing endorphins. In animal breeding systems both solid and liquid components may be treated with the energized liquid so as to enhance the quality of e.g. meat and milk. In nutriceuticals, e.g. for egg laying purposes, energized water may be added so as to enhance the quality and the production of specific amino acids and fatty acids. In irrigation systems in the fields of agriculture or reforestation energized water may be added in order to increase the water quality and thus the growth and yield factors and the quality of the crops. The invention may also be utilized in the fish industry, where water is used as a prime cleansing liquid. Here the energized water may be added in the rinsing, washing and bathing process so as to increase the quality and the shelf life of the products treated. Furthermore the energized water may be used in aquariums and swimming pools as well as in similar facilities. The energized water may then be added directly as a solution or indirectly by using a filter process, e.g. using a cassette tube of the type shown in FIGS. 2 and 3. It may also be mentioned that energized water according to the invention may be used for ice-producing purposes. The invention may also be utilized in processes for manufacturing polymer products, such as plastic containers and plastic bags for the storage for food or other materials with a limited shelf life. In this connection the resin for the manufacture is the liquid which is energized. Furthermore the liquid treated according to the invention may be used in connection with the manufacture of preparates for skin and health care as well as for hair and nail products. The invention may also be utilized in industrial cooling systems, such as boiler feed systems, cooling towers, coolants for machinery and humidification systems.

Due to the technique used in accordance with the invention, the energy levels and frequencies obtained are up to and including also nano ratings never experienced in this technological field before.

1-7. (canceled)

8. Apparatus for treatment of fluids comprising a tube, a first cone arranged at one end of the tube, said first cone forming an inlet opening of the apparatus, a second cone arranged at the other end of said tube, said second cone forming an outlet opening of the apparatus and means for generating a rotating flow of incoming fluid positioned inside side first cone, characterised in that the interior of said tube contains a batch of mineral grains having been exposed to a clustered water.

9. Apparatus according to claim 8, characterised in that said means for generating a rotating flow is formed by screw shaped blades.

10. Apparatus according to claim 8, characterised in that said mineral grains is a piezoelectric quartz.

11. Method of treatment of fluids comprising the following method steps:
exposing mineral grains to a clustered fluid,
positioning a batch of said mineral grains into an apparatus for treatment of fluids comprising a tube, a first cone arranged at one end of the tube, said first cone forming an inlet opening of the apparatus, a second cone arranged at the other end of said tube, said second cone forming an outlet opening of the apparatus and means for generating a rotating flow of incoming fluid positioned inside side first cone, and
passing a fluid through said apparatus.

12. Method according to claim 8, characterised in that said fluid is water.
